(19) United States
(12) Patent Application Publication
(10) Pub. No.: US 2011/0294712 A1
(43) Pub. Date: Dec. 1, 2011

(54) DISPENSER FOR ON-DEMAND GENERATION OF SANITIZING SOLUTION

(76) Inventor: Ashok V. Joshi, Salt Lake City, UT (US)

(21) Appl. No.: 12/789,123

(22) Filed: May 27, 2010

(51) Int. Cl. A61K 8/20; B67D 7/08; A61Q 19/10

(52) U.S. Cl. 510/131; 222/52

(57) ABSTRACT

A dispenser to generate and dispense a cleaning agent on demand. The dispenser includes a supply chamber, an agent generation cell, and a dispensing actuator. The supply chamber stores an alkali halide compound. The agent generation cell is coupled to the supply chamber and includes a porous ionic conducting membrane to separate ion components of the alkali halide compound. The ion components include alkaline ions and halide ions. The dispensing actuator is coupled to the agent generation cell. The dispensing actuator activates the agent generation cell on-demand to generate and dispense the cleansing agent to a user in response to an interaction between the user and the dispensing actuator. The cleansing agent includes the halide ions from the alkali halide compound and is substantially free of the alkaline ions.

### BACKGROUND

Sanitizing solutions that use alcohol as the sanitizing agent are used in a wide variety of applications. Common surfaces often require cleansing or sanitizing. However, as alcohol-based sanitizing solutions evaporate, greenhouse and other noxious gases are formed. It would be an advancement to provide an apparatus for generating and dispensing cleansing, sanitizing, and antimicrobial solutions on-demand that does not form greenhouse and other noxious gases.

Hypochlorous acid (HOCl, also known as chloric(I) acid) is a weak acid. Hypochlorous acid is used as a bleach, an oxidizer, a deodorant, and a disinfectant. Hypochlorous acid has been approved by the U.S. Food and Drug Administration for use in food washing and cleaning applications. Hypochlorous acid has also been approved for use on human skin as a cleansing and sanitizing solution. Hypochlorous acid is also used in some applications to promote wound healing.

The addition of chlorine to water can result in both hypochlorous acid and hydrochloric acid (HCl):

Cl₂+H₂O→HOCl+HCl

However, hypochlorous acid rapidly breaks down and, hence, has a relatively short life. More specifically, hypochlorous acid cannot be isolated in pure form due to rapid equilibration with the hypochlorite anion (OCl⁻) and hydrogen ion (H⁺):

HOCl [symbol] OCl⁻+H⁺

Consequently, it is difficult to store hypochlorous acid for use in sanitization applications because the chemical composition of hypochlorous acid changes very quickly.

### SUMMARY

Embodiments of an apparatus are described. In one embodiment, the apparatus is a dispenser to generate and dispense a cleaning agent on demand. The dispenser includes a supply chamber, an agent generation cell, and a dispensing actuator. The supply chamber stores an alkali halide compound. The agent generation cell is coupled to the supply chamber and includes a porous ionic conducting membrane to separate ion components of the alkali halide compound. The ion components include alkaline ions and halide ions. The dispensing actuator is coupled to the agent generation cell. The dispensing actuator activates the agent generation cell on-demand to generate and dispense the cleansing agent to a user in response to an interaction between the user and the dispensing actuator. The cleansing agent includes the halide ions from the alkali halide compound and is substantially free of the alkaline ions. Other embodiments of the apparatus are also described.

Embodiments of a system to generate and dispense a cleansing agent are also described. In one embodiment, the system includes an agent generation cell, a dispensing actuator, and a controller. The agent generation cell receives water and an alkali halide compound. The agent generation cell includes a porous ionic conducting membrane to separate alkaline ions and halide ions of the alkali halide compound for generation of the cleansing agent The cleansing agent includes a solution of the water and a halide-based compound that is substantially free of the alkaline ions. The dispensing actuator facilitates a user interaction so that the user can initiate generating and dispensing the cleansing agent. The controller activates the agent generation cell to generate the cleansing agent in response to the user interaction with the dispensing actuator. Other embodiments of the system are also described.

Embodiments of a method for on-demand generation of a sanitizing solution with a halide cleansing agent are also described. In one embodiment, the method includes applying an electrical potential difference across an anode and a cathode on opposite sides of a porous ionic conducting membrane in response to a user interaction with a dispenser for the sanitizing solution. The method also includes separating ions from an alkali halide compound to form alkaline cations and halide anions. The method also includes generating an aqueous solution with the halide cleansing agent from the halide anions and water, wherein the aqueous solution is substantially free of alkaline constituents. Other embodiments of the method are also described.

Embodiments of another apparatus are also described. In one embodiment, the apparatus is a dispenser to generate and dispense a cleaning agent using an electrolyte which contains a noble metal halide. One example of a suitable noble metal halide is silver chloride (AgCl), although other embodiments may use other noble metal halides. The dispenser includes a supply chamber to store water. The dispenser also includes an agent generation cell coupled to the supply chamber. The agent generation cell includes the electrolyte and first and second porous electrodes on opposite sides of the electrolyte. The dispenser also includes a dispensing actuator coupled to the agent generation cell. The dispensing actuator activates the agent generation cell on-demand to generate and dispense the cleansing agent to a user in response to an interaction between the user and the dispensing actuator. In some embodiment, the cleansing agent includes a halide based acid. As one example, the noble metal halide may be silver chloride (AgCl), and the resulting cleansing agent may be hypochlorous acid. Other embodiments of the apparatus are also described.

Some embodiments may combine two or more of the various structures described herein. Other embodiments of the exhaust sensor are also described. Other aspects and advantages of embodiments of the present invention will become apparent from the following detailed description, taken in conjunction with the accompanying drawings, illustrated by way of example of the principles of the invention.

### BRIEF DESCRIPTION OF THE DRAWINGS

FIG. 1 is a schematic diagram of an embodiment of a sanitizing solution dispenser to produce a hypochlorous acid sanitizing solution.

FIG. 2 is a schematic diagram of another embodiment of a sanitizing solution dispenser to produce a chloride dioxide sanitizing solution.

FIG. 3 is a schematic diagram of another embodiment of a sanitizing solution dispenser to produce a hypochlorous sanitizing solution.

FIGS. 4A-C are schematic diagrams of other embodiments of sanitizing solution dispensers which use tubular ionic conducting membranes.

FIG. 5 is a schematic diagram of one embodiment of a sanitizing solution system to generate and dispense sanitizing solution on-demand.

FIG. 6 is a schematic diagram of one embodiment of a cartridge system for use with the sanitizing solution system of FIG. 5.

FIG. 7 is a schematic block diagram of another embodiment of a sanitizing solution system to generate and dispense a sanitizing solution on demand.

FIG. 8 is a flow chart diagram of one embodiment of a method of operation for generating and dispensing a sanitizing solution on demand.

FIG. 9 is a schematic block diagram of a sanitizing solution dispenser to produce a hypochlorous acid sanitizing solution using an electrolyte that contains silver chloride (AgCl).

FIG. 10 is a schematic block diagram of one embodiment of a sanitizing solution dispenser to produce a hypochlorous acid sanitizing solution using an electrolyte that contains silver chloride (AgCl).

FIG. 11 is a schematic block diagram of one embodiment of a system to purify water.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments of a dispenser are described herein, at least some of the described embodiments provide an electrochemical cell and process for producing a halide based sanitizing solution from readily available and environmentally safe starting materials, or feedstock. Some examples of starting materials include sodium chloride (NaCl) and sodium chlorate (NaClO₃). Other embodiments may use other alkali halide compounds such as sodium bromide (NaBr), sodium iodide (NaI), and so forth. When combined with water (H₂O) and a process for separating the alkaline and halide ions, the dispenser can generate a halide based sanitizing solution that does not include alkaline constituents, or excludes substantially all of the alkaline ions. Some examples of resulting sanitizing solutions include hypochlorous acid (HOCl) and chlorine dioxide (ClO₂) as the cleansing agent with and aqueous solution. Other embodiments may generate other halide based sanitizing solutions. For example, the sanitizing solution may include HO“X”+H₂O+“X”, where X is some element-containing water. Alternatively, X may be some compound-containing water. In other words, there may be un-reacted elements and/or compounds left in solution in the water. In some embodiments, X may be one or more of chlorine, bromine, and iodine.

Hypochlorous acid (HOCl) may be prepared with an electrolytic process using a cationic or an anionic conductive membrane. In the cationic process, sodium ions are drawn out of the solution containing water and sodium chloride. The remaining chloride ions react with the water to form a solution of hypochlorous acid. Because hypochlorous acid is not stable for long-term storage, the ability to generate these compounds at suitable concentrations at the point of use make it more likely that such environmentally friendly compounds can be used.

In general, the cationic conducting membrane is capable of selectively transporting specific cations (e.g., Na⁺) between solutions on either side of the cationic conducting membrane. Some examples of cationic conducting membranes that are suitable for use with embodiments described herein include any known or novel type of sodium super-ionic conductor (NaSICON) membrane (including, but not limited to NaSICON-type membranes produced by Ceramatec Inc. of Salt Lake City, Utah), lithium super-ionic conductor (LiSICON) membranes, potassium super-ionic conductor (KSICON) membranes, and other polymeric cationic conducting membranes (such as NAFION® membranes produced by DuPont). For convenience, general references to MSICON membranes may be used to collectively or generically refer to a membrane that is capable of selectively transporting M ions, where M is lithium, sodium, and/or potassium. More generally, in some embodiments, the cationic conducting membrane can be any material with minimal sodium conductivity. Also, the cationic conducting membrane may be a porous or dense solid material. Other embodiments may use a sodium-based glass membrane or another type of sodium (or other alkali-based) membrane.

In the anionic process, chloride ions are extracted from a solution containing chloride ions, such as sodium chloride. The extracted chlorine ions react with water on the other side of the membrane to form a solution of hypochlorous acid. In some embodiments, the anionic exchange membrane is a polymeric membrane which functions at approximately room temperature, or other relatively low temperatures. Some examples of anionic exchange membranes include, but are not limited to, AMX, ACS (Neosepta, Japan), AMI-7001 (Ultrex, USA), MA-40 (PO Stchekin, Russia). Other examples include AFN, ACS, AMX, PCA35, PCA100, PCA200D, AHM, ACM, AM-1, AM-3, and other similar anionic exchange membranes.

Instead of generating hypochlorous acid, chlorine dioxide (ClO₂) may be prepared with an electrolytic process using a cationic conductive membrane. The cationic process includes extracting sodium cations from an aqueous sodium chlorate solution. The removal of the sodium cations allows the remaining solution to form an aqueous chlorine dioxide solution.

These and equivalent processes result in an effective sanitizing or cleansing agent that is safe for humans, as well as the environment. Additionally, some embodiments of the sanitizing solution further include alcohol for additional cleansing properties, although these embodiments may have environmental effects.

In some embodiments, the dispenser uses an electrochemical generation cell to generate the hypochlorous acid, chlorine dioxide, or other cleansing agent. The electrochemical cell can be configured in a variety of ways to produce the sanitizing solution. In one embodiment, an anode is disposed in an anionic chamber, and a cathode is disposed in a corresponding cationic chamber. The anions or cations move across the membrane when voltage is applied across the anode and cathode electrodes of the electrochemical cell.

The sanitizing solution dispenser may be configured in numerous forms depending on the on-demand application for the sanitizing solution. Examples of such forms include, but are not limited to, automatically and mechanically activated dispensers. Specific applications may include public sanitizing applications in hospitals, hotels, schools, ocean vessels, and in applications where cleansing, sanitizing, or antimicrobial solutions are used.

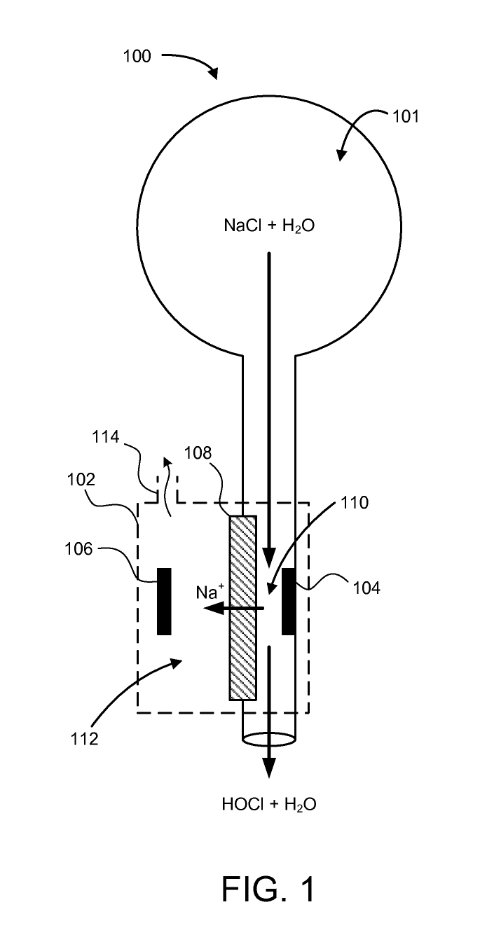

FIG. 1 is a schematic diagram of an embodiment of a sanitizing solution dispenser 100 to produce a hypochlorous acid sanitizing solution. The illustrated dispenser 100 includes a supply chamber 101. The supply chamber 101 contains an aqueous solution that includes sodium chloride (NaCl) and water (H₂O). The supply chamber 101 supplies the aqueous sodium chloride solution to an agent generation cell 102 which generates and dispenses a cleansing agent. The supply chamber 101 may be separated from the agent generation cell 102 by a one-way valve (not shown) or other type of back flow prevention device. In the illustrated embodiment, the cleansing agent is hypochlorous acid (HOCl) in an aqueous solution with water. In other embodiments, other comparable starting materials may be used (e.g., NaBr and H₂O or NaI and H₂O) to produce a comparable cleansing agent (e.g., hypobromous acid (HOBr) or hypoiodous acid (HOI)) or bromine-containing water or iodine-containing water.

In order to generate the cleansing agent solution, the agent generation cell 102 includes electrochemically active electrodes 104 and 106 located either side of a cationic conducting membrane 108. Although the electrical connections for the electrodes 104 and 106 are not shown, one skilled in the art will understand how to implement such connections in light of the description herein. In particular, the anode 104 is on one side of the cationic conducting membrane 108, and the cathode 106 is on an opposite side of the cationic conducting membrane 108. When a voltage difference is applied to the anode 104 and the cathode 106, the cationic conducting membrane 108 selectively transfers cations away from the anode 104 and toward the cathode 106. In the illustrated embodiment, the cationic conducting membrane 108 removes sodium cations (Na⁺) from the sodium chloride solution supplied from the supply chamber 101. The dissociation of the sodium cations from the chloride anions in the aqueous solution generates hypochlorous acid (HOCl) in a solution with water. Thus, the resulting aqueous hypochlorous acid solution can be used for sanitizing or cleansing. In one embodiment, the sanitizing solution is dispensed to a user through a discharge opening.

For reference in describing the dispenser 100, the area in which the anode 104 is disposed is generally referred to as the generation chamber 110. Some embodiments of the generation chamber 110 permit the agent generation cell 102 to hold a predetermined volume of solution. The predetermined volume of solution may correspond to a portion of a dose of sanitizing solution, approximately one dose of sanitizing solution, or more than one dose of sanitizing solution. As a specific example, one dose of sanitizing solution may include between approximately 1.0 to 5.0 cubic centimeters (cc). In another specific example, one dose of sanitizing solution may include between approximately 1.0 to 8.0 cc. Other embodiments of the generation chamber 110 permit continuous flow of the supply solution into the generation chamber 110 and/or continuous flow of the generated sanitizing solution out of the generation chamber 110, depending on the types of valves or other fluid controls implemented within the agent generation cell 102.

Also, the area in which the cathode 106 is disposed is generally referred to as the collection chamber 112 of the illustrated embodiment because that is where the extracted sodium cations are contained or scrubbed. In some embodiments, the material collected in the collection chamber 112 is discarded. In other embodiments, the material collected in the collection chamber 112 is retrieved and recycled. In some embodiments, the collection chamber 112 also includes a vent 114 to allow hydrogen (H₂), or another gaseous chemical composition, to escape from the collection chamber 112, depending on the type of chemical compositions that are contained and/or generated within the collection chamber 112.

In an alternative embodiment, the collection chamber 112 includes an amount of conditioning agent to react with the sodium cations (Na⁺). As one example, the conditioning agent may include zinc chloride (ZnCl₂) to result in a combination of zinc (Zn) and sodium chloride (NaCl). In this example, it may be acceptable to forego venting through the vent 114. As another example, the conditioning agent may include zinc carbonate (ZnCO₃). Other embodiments may use other conditioning agents.

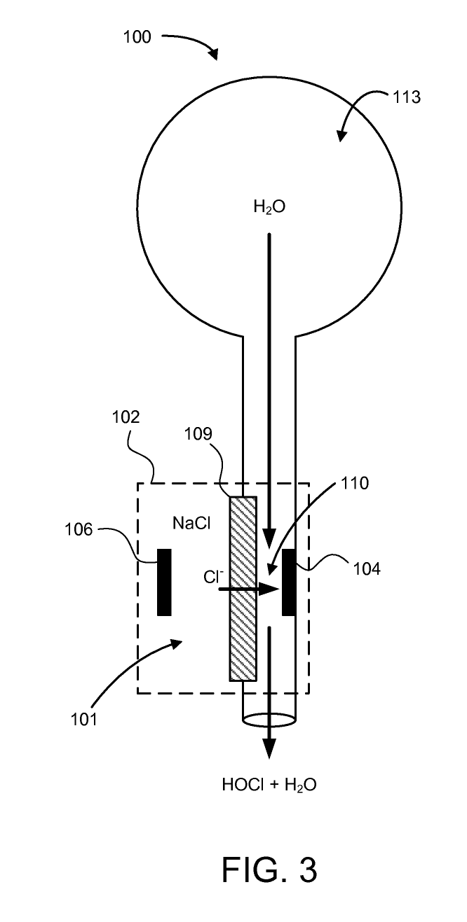

Although not shown, further embodiments of the dispenser 100 may utilize a different number of chambers and/or fluid channels. For example, in some embodiments, the sodium chloride and water is supplied from separate supply chambers. Furthermore, the locations of the various chambers may be different in other embodiments, depending on the type of function or purpose that each chamber serves. For example, in some embodiments, the area in which the cathode 106 is disposed may be designated as a supply chamber 101 if the alkali halide compound is supplied from or contained in that space (refer to FIG. 3).

Also, although various types of cleansing agents may be used within the sanitizing solution, and the corresponding chemical characteristics may vary, at least some of the sanitizing solutions are generated to have a controlled pH value. In some embodiment, the pH value is within the range of approximately 6.5 to 10.0. In another embodiment, the pH value of the sanitizing solution is within the range of approximately 7.0 to 9.5. Other embodiments may control the pH range of the sanitizing solution with less or more tolerance.

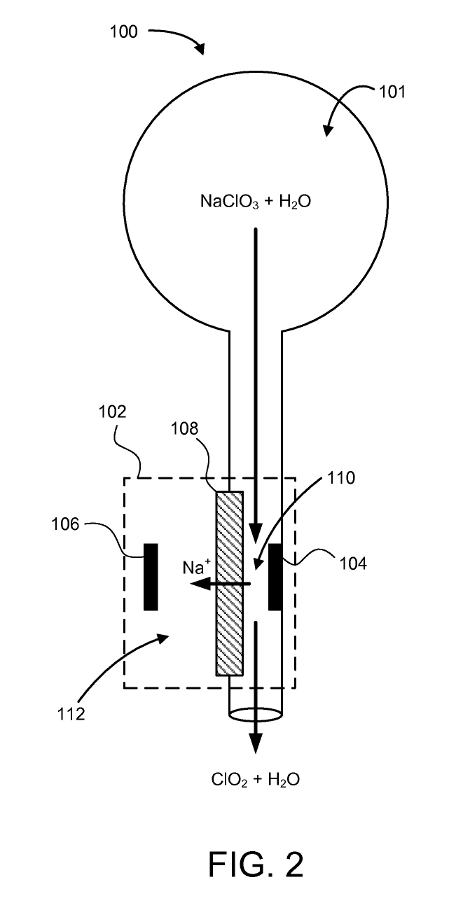

FIG. 2 is a schematic diagram of another embodiment of a sanitizing solution dispenser 100 to produce a chlorine dioxide sanitizing solution. The dispenser 100 of FIG. 2 is substantially similar to the device 100 of FIG. 1 described above. However, in the illustrated embodiment, the starting materials stored in the supply chamber 101 are different, and the composition of the generated sanitizing solution is consequently different. In other embodiments, other comparable starting materials may be used (e.g., NaBrO₃ and H₂O) to produce a comparable cleansing agent (e.g., bromine dioxide (BrO₂) and H₂O).

In the illustrated embodiment, the supply chamber 101 contains an aqueous solution that includes sodium chlorate (NaClO₃) and water (H₂O). The supply chamber supplies the aqueous sodium chlorate solution to the agent generation cell 102 which generates and dispenses a sanitizing solution of chlorine dioxide (ClO₂) and water. One or more back flow prevention devices such as a one-way valve may be positioned between the supply chamber 101 and the agent generation cell 102.

In a similar manner as described above, the cationic conducting membrane 108 selectively transfers sodium cations from the generating chamber 110 to the collection chamber 112. The dissociation of the sodium cation from the chloride anions in the aqueous solution generations the chlorine dioxide in the solution with water. The generated sanitizing solution is dispensed through the discharge opening for use in various sanitizing or cleansing activities.

FIG. 3 is a schematic diagram of another embodiment of a sanitizing solution dispenser 100 to produce a hypochlorous acid sanitizing solution. Although the dispenser 100 of FIG. 3 is similar in some aspects to the dispensers 100 of FIGS. 1 and 2, it should be noted that the supply chamber 101 is located in a different position relative to the agent generation cell 102. Specifically, the supply chamber 101 is located at or near the cathode 106. Additionally, the dispenser 100 includes a supplemental supply chamber 113 that is separate from the supply chamber 101.

In the illustrated embodiment, sodium chloride is contained in the supply chamber 110, and water is contained in the supplemental supply chamber 113. As in other embodiments, one or more back flow prevention devices such as a one-way valve may be positioned between the supplemental supply chamber 113 and the agent generation cell 102.

When water is supplied to the generation chamber 110 of the agent generation cell 102, an anionic conducting membrane 109 (instead of a cationic conducting membrane 108) selectively transports chloride anions (Cl⁻) from the supply chamber 112 to the generation chamber 110. The chloride anions react with the water in the generation chamber 110 to form an aqueous solution of hypochlorous acid, similar to the device 100 of FIG. 1.

Thus, the device 100 of FIG. 3 implements an alternative embodiment that uses an anionic conducting membrane 109 to transport chloride anions, rather than a cationic conducting membrane to transport sodium cations. In some embodiment, the device 100 of FIG. 3 may be advantageous over other embodiments because a user simply supplies additional water to the supplemental supply chamber 113, rather than supplying a pre-mixed aqueous sodium chloride solution.

In alternative embodiments, another type of halide salt may be used, instead of NaCl. For example, the composition within the supply chamber 101 may include magnesium (Mg), calcium (Ca), iron (Fe), nickel (Ni), silver (Ag), copper (Cu), barium (Ba), or another similar material, instead of sodium (Na). As a more specific example, the composition within the supply chamber 101 may be magnesium chloride (MgCl₂). As another specific example, the composition within the supply chamber 101 may be magnesium hydroxide (Mg(OH)₂). Other embodiments may use other specific halide salt compositions.

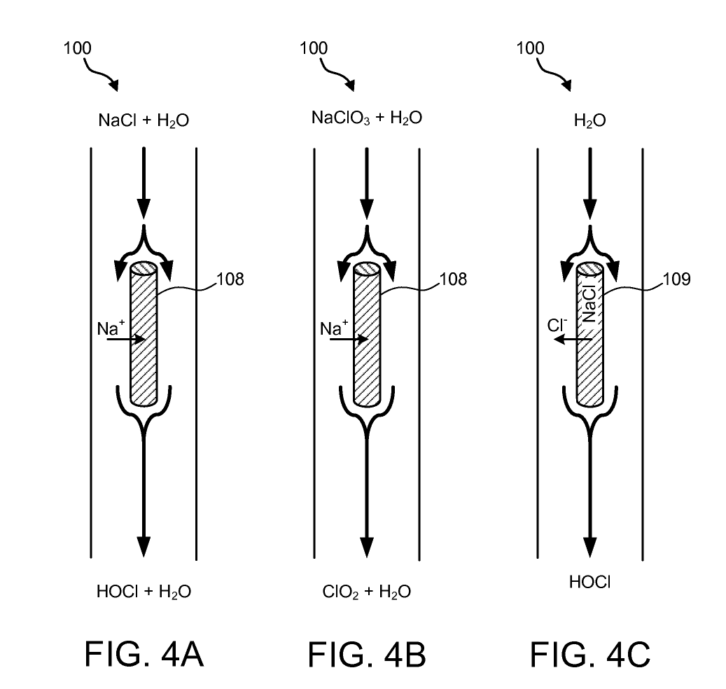

FIGS. 4A-C are schematic diagrams other embodiments of sanitizing solution dispensers 100 which use tubular ionic conducting membranes 109. For reference, the dispensers 100 of FIGS. 4A, 4B, and 4C functionally correspond to the dispensers 100 shown in FIGS. 1, 2, and 3.

FIG. 4A depicts a tubular cationic conducting membrane 108. In one embodiment, a collection chamber 112 (not shown in FIG. 4A) is defined within the tubular cationic conducting membrane 108 so that the sodium cations that are removed from the aqueous sodium chloride solution can be contained within the tubular cationic conducting membrane 108. The functionality of the tubular cationic conducting membrane 108 of FIG. 4B is similar to the functionality of the tubular cationic conducting membrane 108 of FIG. 4A. As mentioned above, other halides instead of chlorine could be used within the starting materials and/or the resulting cleansing agent.

In the illustrated embodiment of FIG. 4C, the dispenser 100 includes a tubular anionic conducting membrane 109. Although not illustrated, the tubular anionic conducting membrane 109 may contain a supply of sodium chloride within a supply chamber 101 within the tubular structure.

Other embodiments of the cationic and anionic conducting membranes 108 and 109 may be other shapes, sizes, thicknesses, and so forth. For example, some embodiments of the cationic and anionic conducting membranes 108 and 109 are planar.

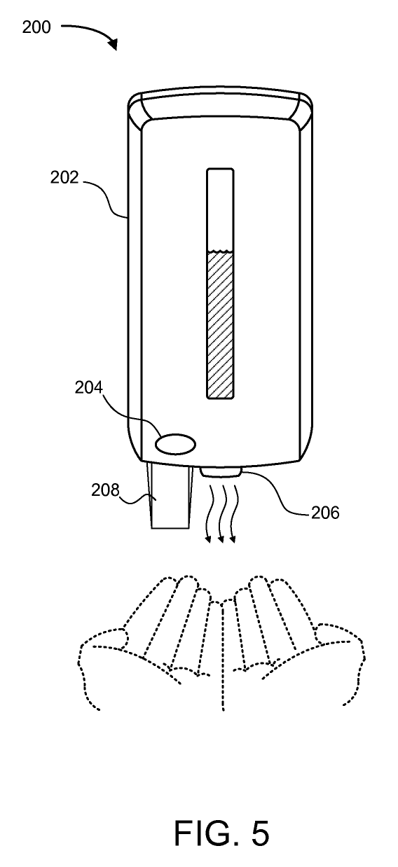

FIG. 5 is a schematic diagram of one embodiment of a sanitizing solution system 200 to generate and dispense sanitizing solution on-demand. In the illustrated embodiment, the sanitizing solution system 200 includes a housing 202 to contain various internal components. For example, the housing 202 may include the supply chamber 101, the agent generation cell 102, corresponding circuitry and mechanical structures, and other components described herein. In one embodiment, the housing 202 mounts to wall brackets (not shown) or directly to a wall or other vertical surface. Other embodiments include a desk/table/floor mounted, or ceiling mounted, or free-standing structure. Other embodiments of the housing 202 may be mounted or supported in other ways.

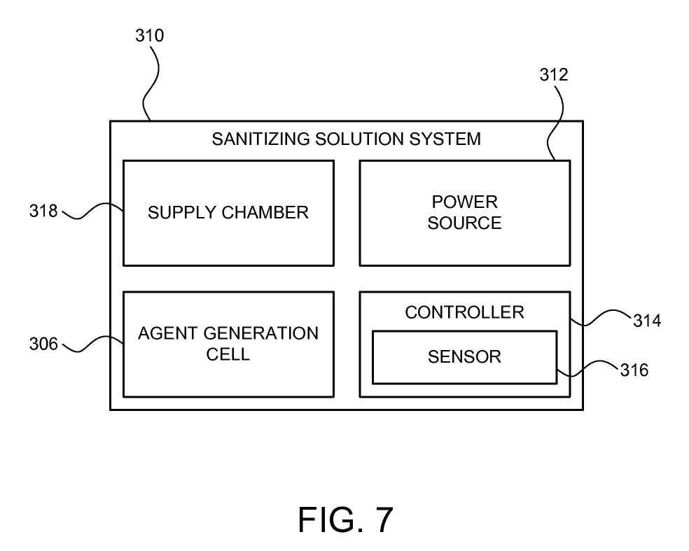

The illustrated embodiment also includes a proximity detector 204 that is integrated with the housing 202. In general, the proximity detector 204 is an automated detection mechanism to determine when a user (the user's hands are shown dashed) is in position to receive a dose of the sanitizing solution. Upon detection, the proximity detector 204 may generate one or more signals which are communicated to a controller (refer to FIG. 7), and the controller controls the operations of the agent generation cell 102 to dispense the dose through a dispensing port 206. In the depicted embodiment, the proximity detector 204 is on the front panel of the housing 202 and the dispensing area is below the dispensing port 206, but other embodiments may have different configurations.

In one embodiment, the agent generation cell 102 within the housing 202 does not generate a dose of sanitizing solution with the cleansing agent until the user is detected within the dispensing area. However, some embodiments of the sanitizing solution system 200 may pre-load a volume of the aqueous sodium chloride solution into the generation chamber 110 in anticipation of the next dispensing cycle. Alternatively, some embodiments of the sanitizing solution system 200 do not pre-load the aqueous sodium chloride solution into the generation chamber 110, but rather perform all of the operation on-demand upon detection of the user. Thus, in light of the description herein one skilled in the art will understand that there are various ways to implement the timing and flow of the solutions within the sanitizing solution system 200.

In another embodiment, the sanitizing solution system 202 is activated by a user interaction with a mechanical dispensing actuator such as a lever 208 or button (not shown). Other types of electronic and/or mechanical actuators may be implemented to operate the sanitizing solution system 200.

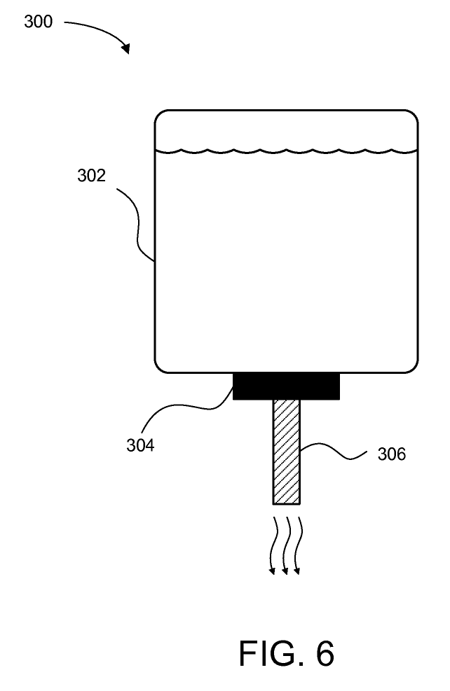

FIG. 6 is a schematic diagram of one embodiment of a cartridge system 300 for use with the sanitizing solution system 200 of FIG. 5. The illustrated cartridge system 300 includes a reservoir 302, a coupler 304, and an agent generation cell 306. In some embodiments, the reservoir 302 is plastic and forms watertight supply chamber 101. The plastic is non-reactive with respect to the supply solutions (e.g., H₂O+NaCl or H₂O+NaClO₃ or H₂O). Other non-reactive materials such as glass or ceramic also may be used.

The coupler 304 is coupled to an outlet of the reservoir 302. In the depicted embodiment, the coupler 304 is a ring. In other embodiments, the coupler 304 may have another geometry, including a non-circular geometry. In some embodiments, the coupler 304 includes electrical connections to provide power to the anode and cathode elements 104 and 106 within the agent generation cell 306. Additionally, the coupler 304 may supply power to the proximity sensor 204, a pump (not shown), and/or other sensors (not shown). In some embodiments, the coupler 304 secures the cartridge system 300 within the housing 202 of the sanitizing solution system 200.

The agent generation cell 306 is similar in functionality to one or more of the agent generation cells 102 described above with reference to FIGS. 1-3. In one embodiment, the agent generation cell 306 is coupled to the reservoir 302 via the coupler 304. As described above, the agent generation cell 306 facilitates the generation and dispensing of the sanitizing solution. In some embodiments, the motion of the solution through the cartridge system 300 is induced by gravity. In other embodiments, the solution is pumped through the cartridge system 300 by manual and/or automated pumps (not pictured).

FIG. 7 is a schematic block diagram of another embodiment of a sanitizing solution system 310 to generate and dispense a sanitizing solution on demand. The illustrated sanitizing solution system 310 includes the agent generation cell 306, a power source 312, a controller 314, and a feeder 318. In some embodiments, the power source 312 is a battery or other direct current (DC) source. Alternatively, the power source 312 may be cord and plug for connection to a wall outlet or other alternative current (AC) source. In another embodiment, the power source 312 may be a mechanically actuated power generator that generates electrical power from a motion or force exerted by a user at the sanitizing solution system 200, for example, by actuation of the mechanical lever 208.

In general, the controller 314 controls a generation rate for generating the sanitizing solution with the cleansing agent that is produced by the agent generation cell 306. Additionally, the controller 314 may detect feedback parameters related to the generation of the sanitizing solution with the cleansing agent. In one embodiment, the controller 314 detects the parameters of the system 310 through a sensor 316. The sensor 316 may be configured to detect the state and/or quality of the sanitizing solution and/or the supply solution at the agent generation cell 306.

In some embodiments, the controller 314 resupplies the supply solution in the reservoir 302 of FIG. 6 by activating the feeder 318, which may be connected to an external source such as a water line. Additionally, the controller 314 may be configured to monitor or control other features or functions of the generating and dispensing system 310.

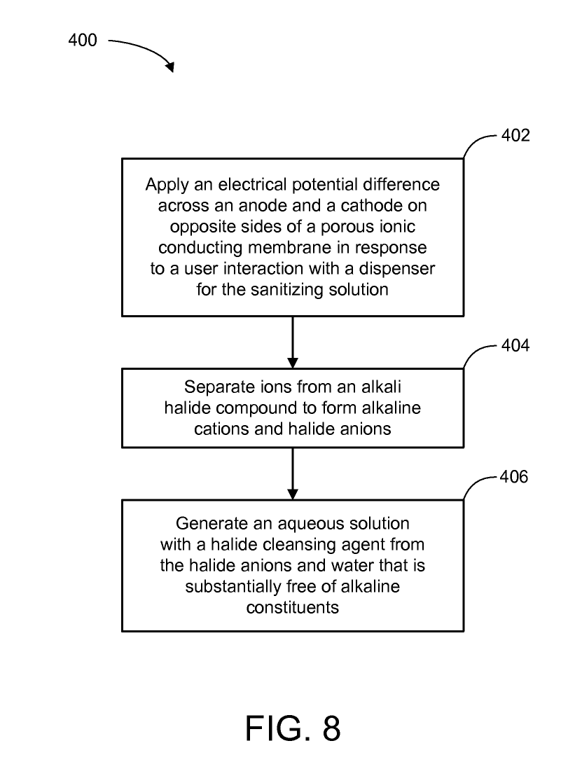

FIG. 8 is a flow chart diagram of one embodiment of a method 400 of operation for generating and dispensing a sanitizing solution on demand. Although the method 400 is described in conjunction with the sanitizing solution systems described above, embodiments of the method 400 may be implemented with other types of sanitizing solution systems.

The illustrated method 400 includes applying 402 an electrical potential difference across the anode 104 and the cathode 106 on opposite sides of the porous ionic conducting membrane 108 or 109 in response to a user interaction with a dispenser for the sanitizing solution. Applying the electrical potential difference in this manner results in separating 404 ions from the alkali halide compound within the generation chamber 110 to form alkaline cations and halide anions. The resulting combination of halide anions and water facilitates generating 406 an aqueous solution with a halide cleansing agent (e.g., HOCl or ClO₂) from the halide anions and water. Moreover, the aqueous solution is substantially free of alkaline constituents because the alkali cations (e.g., Na⁺) are contained in the collection chamber 112. The depicted method 400 then ends.

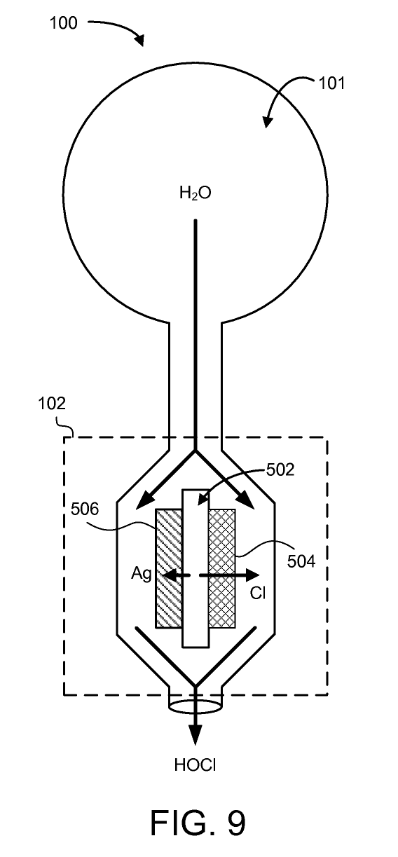

FIG. 9 is a schematic block diagram of a sanitizing solution dispenser 100 to produce a hypochlorous acid sanitizing solution using an electrolyte 502 that contains silver chloride (AgCl). Although the illustrated embodiment is specifically described with reference to the AgCl electrolyte 502, other embodiments may use other similar electrolytes such as AgBr, AgI, and so forth. In a general embodiment, the electrolyte may be any type of electrolyte that contains a noble metal halide composition.

The illustrated dispenser 100 stores H₂O in the supply chamber 101 and/or the dispensing channel until the dispenser 100 is activated by a user. Upon activation, H₂O in the agent generation cell 102 is chlorinated by emission of a chlorine gas from the AgCl electrolyte 502. In one embodiment, a back flow prevention device is positioned between the supply chamber 101 and the agent generation cell 102. In particular, a dissipation voltage is applied to the porous electrodes 504 and 506 on either side of the AgCl electrolyte 502. The dissipation voltage may depend on several factors. In some embodiments, the dissipation voltage is between about 1 to 20 volts. In other embodiments, the dissipation voltage is between about 1 to 12 volts. In other embodiments, the dissipation voltage is between about 1.2 to 1.5 volts. Other embodiments may use other dissipation voltage ranges or levels.

In the illustrated embodiment, the Cl dissociates as a gas and passes through the first porous electrode 504, thus dissolving in the H₂O to form HOCl or chlorine-containing water. The Ag dissociates as a solid and is captured in the second porous electrode 506. To the extent that some of the Ag enters the stream of H₂O, the amount of Ag would be relatively small and may contribute to the cleansing ability of the agent. The cleansing agent could also include silver chloride-containing water.

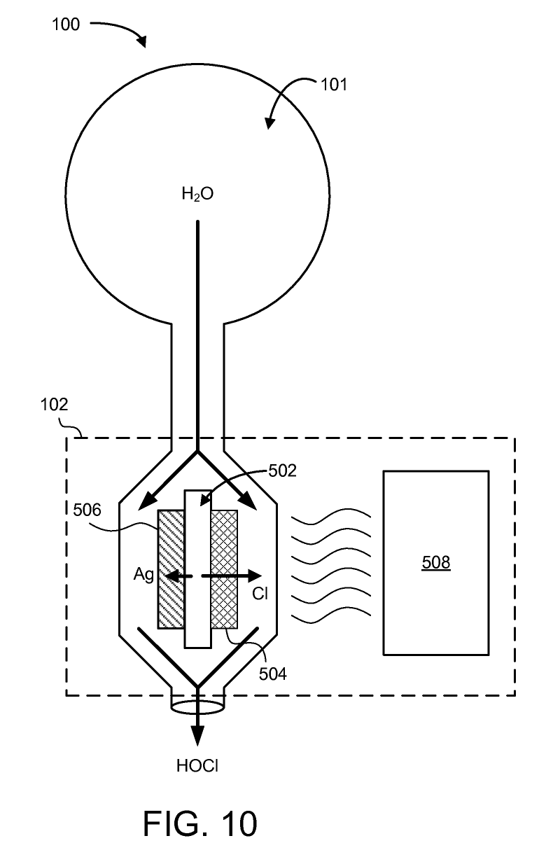

FIG. 10 is a schematic block diagram of one embodiment of a sanitizing solution dispenser 100 to produce a hypochlorous acid sanitizing solution using an electrolyte 502 that contains silver chloride (AgCl). Although the illustrated embodiment is specifically described with reference to AgCl as the electrolyte 502, other embodiments may use other similar electrolytes such as AgBr, AgI, and so forth. In a general embodiment, the electrolyte 502 may be any type of electrolyte that contains a noble metal halide composition.

The illustrated dispenser 100 stores H₂O in the supply chamber 101 and/or the dispensing channel until the dispenser 100 is activated by a user. Upon activation, H₂O in the agent generation cell 102 is chlorinated by emission of a chlorine gas from the AgCl electrolyte 502. In the illustrated embodiment, ultraviolet (UV) radiation is emitted from an UV emitter 508. The radiation is directed to the porous electrodes 504 and 506 on either side of the AgCl electrolyte 502. The wavelength of the radiation may depend on several factors. In some embodiments, the emitted radiation is between about 180 to 640 nm. Other embodiments may use other wavelengths.

In the illustrated embodiment, the chlorine dissociates as a gas and passes through the first porous electrode 504, thus dissolving in the H₂O to form HOCl. The Ag dissociates as a solid and is captured in the second porous electrode 506. To the extent that some of the Ag enters the stream of H₂O, the amount of Ag would be relatively small and may contribute to the cleansing ability of the agent.

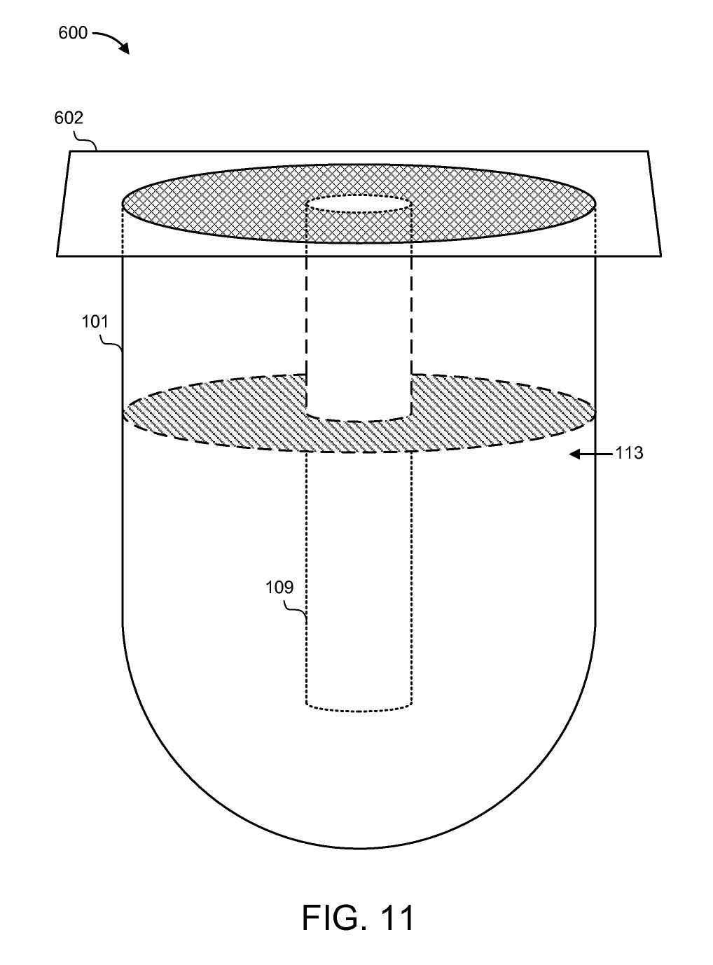

FIG. 11 is a schematic block diagram of one embodiment of a system 600 to purify water 113. In the illustrated embodiment, a supply container 101 contains water 113. In other embodiments, the supply container 101 holds other liquids or materials. The illustrated embodiment also includes a purification cell 109. In some embodiments the purification cell 109 contains silver chloride (AgCl). Other embodiments include other halide-based chemical combinations. Some embodiments of the purification cell 109 contain silver chloride in pellet, powder, solid, liquid, and/or suspended form. In the depicted embodiment, the purification cell 109 can be activated through application of an electrical potential, radiation exposure (for example, UV light), or other application of energy to drive a chemical reaction. In some embodiments, the energy is provided from a fixed energy source such as residential or commercial power sources. In other embodiments, the energy is provided by a portable source, for example, batteries, handcrank generation, portable solar, or other such sources which would improve the mobility of the device.

In the illustrated embodiment, a filter 602 is coupled to a top portion of the supply container 101. The filter 602 may prevent particulate matter from entering into or exiting the supply container 101. In some embodiments, the filter 602 is a mesh or screen. In other embodiments, the filter 602 is a porous membrane. Other embodiments of the filter 602 include other physical filter structures.

It should also be noted that, for the various embodiments described herein, the exact composition of the supply materials in the supply chamber 101 and/or 113 may vary. In some embodiments, the supply material may be stored in a liquid state. In other embodiments, the supply material may include a gelling agent such as a food-grade gelling agent. One example of a food-grade gelling agent is carboximethol cellulose (CMC), although other types of gelling agents may be used in other embodiments.

Also, for the various embodiments described herein, the cleansing agent may be dispensed from the dispenser 100 in various forms. In some embodiments, the cleansing agent may be dispensed as a liquid. In other embodiment, the cleansing agent may be dispensed as a gel. In other embodiments, the cleansing agent may be dispensed as a mist, for example, using an atomizer or other type of mist producer. Other embodiments may dispense the cleansing agent in another form or in a combination of forms (e.g., a gel with suspended solid particles).

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although specific embodiments of the invention have been described and illustrated, the invention is not to be limited to the specific forms or arrangements of parts so described and illustrated. The scope of the invention is to be defined by the claims appended hereto and their equivalents.

What is claimed is:

1. A dispenser to dispense a cleansing agent, the dispenser comprising:
a supply chamber to store an alkali halide compound;
an agent generation cell coupled to the supply chamber, the agent generation cell comprising a porous ionic conducting membrane to separate ion components of the alkali halide compound, wherein the ion components comprise alkaline ions and halide ions; and
a dispensing actuator coupled to the agent generation cell, the dispensing actuator configured to activate the agent generation cell on-demand to generate and dispense the cleansing agent to a user in response to an interaction between the user and the dispensing actuator, wherein the cleansing agent comprises the halide ions from the alkali halide compound and is substantially free of the alkaline ions.

2. The dispenser according to claim 1, wherein:
the alkali halide compound is combined with an aqueous solution within the supply chamber; and
the cleansing agent comprises a halide compound within an aqueous solution.

3. The dispenser according to claim 1, wherein the aqueous solution with the cleansing agent is free of alcohol content.

4. The dispenser according to claim 1, wherein the aqueous solution with the cleansing agent further comprises alcohol.

5. The dispenser according to claim 1, wherein the porous ionic conducting membrane comprises a cationic conducting membrane.

6. The dispenser according to claim 5, wherein:
the alkali halide compound comprises sodium chloride (NaCl) in a solution with water (H₂O); and
the agent generation cell is configured to remove sodium cations (Na⁺) from the solution to generate the cleansing agent comprising hypochlorous acid (HOCl) in a solution with the water (H₂O).

7. The dispenser according to claim 5, wherein:
the alkali halide compound comprises sodium chlorate (NaClO₃) in a solution with water (H₂O); and
the agent generation cell is configured to remove sodium cations (Na⁺) from the solution to generate the cleansing agent comprising chlorine dioxide (ClO₂) in a solution with the water (H₂O).

8. The dispenser according to claim 5, wherein the agent generation cell further comprises:
a generation chamber to contain at least a portion of the alkali halide compound in a solution with water (H₂O) during the separation of the alkaline ions and halide ions; and
a collection chamber separated from the generation chamber by the porous cationic conducting membrane, wherein the collection chamber is configured to contain the alkaline ions subsequent to the separation of the alkaline ions from the halide ions.

9. The dispenser according to claim 1, wherein the porous ionic conducting membrane comprises an anionic conducting membrane.

10. The dispenser according to claim 9, wherein:
the alkali halide compound comprises sodium chloride (NaCl); and
the agent generation cell is configured to remove chloride anions (Cl⁻) from the sodium chloride and add the chloride anions to water (H₂O) to generate the cleansing agent comprising hypochlorous acid (HOCl) in a solution with the water (H₂O).

11. The dispenser according to claim 9, wherein:
the alkali halide compound comprises sodium bromide (NaBr); and
the agent generation cell is configured to remove bromide anions (Br⁻) from the sodium chloride and add the bromide anions to water (H₂O) to generate the cleansing agent comprising hypobromous acid (HOBr) in a solution with the water (H₂O).

12. The dispenser according to claim 9, wherein the agent generation cell further comprises:
a supplemental supply chamber to contain water (H₂O) separate from the alkali halide compound in the supply chamber; and
a generation chamber separated from the supply chamber with the alkali halide compound by the porous anionic conducting membrane, wherein the generation chamber is configured to contain at least a portion of the generated cleansing agent upon movement of the halide ions from the supply chamber to the generation chamber and movement of the water from the supplemental supply chamber to the generation chamber.

13. The dispenser according to claim 1, wherein the cleansing agent is in a solution with water (H₂O), and the solution has a pH value between about 6.5 and 10.0.

14. The dispenser according to claim 1, wherein the cleansing agent is in a solution with water (H₂O), and the solution has a pH value between about 7.0 and 9.5.

15. The dispenser according to claim 1, wherein the porous ionic conducting membrane comprises a substantially planar membrane.

16. The dispenser according to claim 1, wherein the porous ionic conducting membrane comprises a substantially tubular membrane.

17. The dispenser according to claim 1, wherein the dispensing actuator is further configured to dispense approximately 1 to 8 cubic centimeters of an aqueous chlorinated solution, wherein the cleansing agent comprises a chloride component within the aqueous chlorinated solution.

18. The dispenser according to claim 1, wherein the dispensing actuator is further configured to dispense approximately 1 to 5 cubic centimeters of an aqueous chlorinated solution, wherein the cleansing agent comprises a chloride component within the aqueous chlorinated solution.

19. The dispenser according to claim 1, wherein the halide ions comprise ions of a halide selected from a group consisting of chlorine, bromine, and iodine.

20. The dispenser according to claim 1, wherein the dispensing actuator comprises a manual actuator with a contact surface, and the interaction between the user and the dispensing actuator comprises a manual force applied by the user to the contact surface of the manual actuator.

21. The dispenser according to claim 1, wherein the dispensing actuator comprises an automatic actuator with a proximity detector, and the interaction between the user and the dispensing actuator comprises detection of the user within a detection range of the proximity detector.

22. The dispenser according to claim 1, wherein the agent generation cell comprises a generation chamber to contain at least a portion of a dose of the generated cleansing agent for dispensing the cleansing agent to the user.

23. A system to generate and dispense a cleansing agent, the system comprising:
an agent generation cell to receive water and an alkali halide compound, the agent generation cell comprising a porous ionic conducting membrane to separate alkaline ions and halide ions of the alkali halide compound for generation of the cleansing agent, wherein the cleansing agent comprises a solution of the water and a halide-based compound that is substantially free of the alkaline ions;
a dispensing actuator to facilitate a user interaction; and
a controller coupled to the agent generation cell and the dispensing actuator, wherein the controller is configured to activate the agent generation cell to generate the cleansing agent in response to the user interaction with the dispensing actuator.

24. The system according to claim 23, further comprising a supply chamber to store the alkali halide compound.

25. The system according to claim 24, wherein the supply chamber comprises a refillable container.

26. The system according to claim 24, wherein the supply chamber comprises a disposable container.

27. The system according to claim 24, wherein the supply chamber contains an aqueous solution of sodium chloride (NaCl), and the porous ionic conducting membrane comprises a cationic conducting membrane to remove sodium cations (Na⁺) from the sodium chloride to produce the cleansing agent comprising an aqueous solution of hypochlorous acid (HOCl).

28. The system according to claim 27, further comprising:
a generation chamber in which the aqueous solution of hypochlorous acid is generated;
a collection chamber separated from the generation chamber by the cationic conducting membrane, wherein the collection chamber is configured to store the sodium cations removed from the sodium chloride;
a cathode disposed within the collection chamber; and
an anode disposed within the generation chamber.

29. The system according to claim 24, wherein the supply chamber contains an aqueous solution of sodium bromide (NaBr), and the porous ionic conducting membrane comprises a cationic conducting membrane to remove sodium cations (Na⁺) from the sodium bromide to produce the cleansing agent comprising an aqueous solution of hypobromous acid (HOBr).

30. The system according to claim 24, wherein the supply chamber contains an aqueous solution of sodium chlorate (NaClO₃), and the porous ionic conducting membrane comprises a cationic conducting membrane to remove sodium cations (Na⁺) from the sodium chloride to produce the cleansing agent comprising an aqueous solution of chlorine dioxide (ClO₂).

31. The system according to claim 30, further comprising:
a generation chamber in which the aqueous solution of chlorine dioxide is generated;
a collection chamber separated from the generation chamber by the cationic conducting membrane, wherein the collection chamber is configured to store the sodium cations removed from the sodium chlorate;
a cathode disposed within the collection chamber; and
an anode disposed within the generation chamber.

32. The system according to claim 24, wherein the supply chamber contains an aqueous solution of sodium bromate (NaBrO₃), and the porous ionic conducting membrane comprises a cationic conducting membrane to remove sodium cations (Na⁺) from the sodium bromide to produce the cleansing agent comprising an aqueous solution of bromine dioxide (BrO₂).

33. The system according to claim 24, further comprising a supplemental supply chamber, wherein the supply chamber contains sodium halide and the supplemental supply chamber separately contains the water, and the porous ionic conducting membrane is configured to remove halide ions from the sodium halide and add the halide ions to the water within the generation chamber to form an aqueous solution, wherein the halide is one of a list of halides consisting of chlorine, bromine, and iodine.

34. The system according to claim 33, further comprising:
a generation chamber in which the aqueous solution wherein the ionic conducting membrane separates the supply chamber and the generation chamber.

35. The system according to claim 24, further comprising a supplemental supply chamber, wherein the supply chamber contains silver chloride (AgCl₂) and the supplemental supply chamber separately contains the water, and the porous ionic conducting membrane comprises an anionic conducting membrane to remove chloride anions (Cl⁻) from the silver chloride and add the chloride anions (Cl⁻) to the water within the generation chamber to form an aqueous solution of hypochlorous acid (HOCl)
a cathode disposed within the supply chamber; and
an anode disposed within the generation chamber;

36. The system according to claim 24, further comprising a supplemental supply chamber, wherein the supply chamber contains magnesium chloride (MgCl₂) and the supplemental supply chamber separately contains the water, and the porous ionic conducting membrane comprises an anionic conducting membrane to remove chloride anions (Cl⁻) from the magnesium chloride and add the chloride anions (Cl⁻) to the water within the generation chamber to form an aqueous solution of hypochlorous acid (HOCl).

37. The system according to claim 23, wherein the controller is further configured to control a dosage amount of the cleansing agent generated for dispensing to the user.

38. The system according to claim 37, wherein the controller is configured to generate and dispense approximately 1 to 5 cubic centimeters of an aqueous chlorinated solution for each dispensing actuation by the user.

39. A method for on-demand generation of a sanitizing solution with a halide cleansing agent, the method comprising:
applying an electrical potential difference across an anode and a cathode on opposite sides of a porous ionic conducting membrane in response to a user interaction with a dispenser for the sanitizing solution;
separating ions from an alkali halide compound to form alkaline cations and halide anions; and
generating an aqueous solution with the halide cleansing agent from the halide anions and water, wherein the aqueous solution is substantially free of alkaline constituents.

40. The method according to claim 39, wherein generating the aqueous solution with the halide cleansing agent comprises:
removing sodium cations (Na⁺) from an aqueous solution of sodium chloride (NaCl) using a cationic conductive membrane; and
generating an aqueous solution of hypochlorous acid (HOCl).

41. The method according to claim 39, wherein generating the aqueous solution with the halide cleansing agent comprises:
removing sodium cations (Na⁺) from an aqueous solution of sodium chlorate (NaClO₃) using a cationic conductive membrane; and
generating an aqueous solution of chlorine dioxide (ClO₂).

42. The method according to claim 39, wherein generating the aqueous solution with the halide cleansing agent comprises:
removing chloride anions (Cl⁻) from sodium chloride (NaCl) using an anionic conductive membrane;
adding the chloride anions to water; and
generating an aqueous solution of hypochlorous acid (HOCl).

43. The method according to claim 39, further comprising dispensing the aqueous solution with the halide cleansing agent to a user in response to electronic activation of a dispensing actuator.

44. The method according to claim 39, further comprising dispensing the aqueous solution with the halide cleansing agent to a user in response to manual activation of a dispensing actuator.

45. A dispenser to dispense a cleansing agent, the dispenser comprising:
a supply chamber to store water;
an agent generation cell coupled to the supply chamber, the agent generation cell comprising:
an electrolyte containing a noble metal halide;
a first porous electrode on a first side of the electrolyte; and
a second porous electrode on a second side of the electrolyte;
a dispensing actuator coupled to the agent generation cell, the dispensing actuator configured to activate the agent generation cell on-demand to generate and dispense the cleansing agent to a user in response to an interaction between the user and the dispensing actuator, wherein the cleansing agent comprises a halide-based acid.

46. The dispenser of claim 45, wherein the noble metal halide comprises silver chloride (AgCl), wherein the silver chloride (AgCl) dissociates into silver (Ag) and chlorine (Cl) upon application of a dissociation voltage at the first and second porous electrodes.

47. The dispenser of claim 46, wherein the first porous electrode is configured to collect substantially all of the dissociated silver, and the second porous electrode is configured to allow gaseous chlorine to pass through into the water as the water travels through the agent generation cell, resulting in generation of hypochlorous acid.

48. The dispenser of claim 46, wherein the dissociation voltage is between about 1 to 20 volts.

49. The dispenser of claim 46, wherein the dissociation voltage is between about 5 to 20 volts.

50. The dispenser of claim 46, wherein the dissociation voltage is between about 5 to 12 volts.

51. A system to purify water, the system comprising:
a supply chamber to store water;
a purification cell coupled to the supply chamber, the purification cell comprising:
an electrolyte containing a noble metal halide;
a first porous electrode on a first side of the electrolyte; and
a second porous electrode on a second side of the electrolyte;
an activator coupled to the purification cell, the activator configured to activate the purification cell on-demand to purify the water within the supply chamber in response to an interaction between the user and the activator.

52. The system of claim 51, wherein the noble metal halide dissociates into components of noble metal and halide upon application of a dissociation voltage at the first and second porous electrodes, wherein the halide is selected from a group consisting of chlorine, bromine, and iodine.

53. The system of claim 52, wherein the first porous electrode is configured to collect substantially all of the dissociated noble metal, and the second porous electrode is configured to allow the halide to pass through into the water as the water travels through the agent generation cell, resulting in generation of hypochlorous acid.

54. The system of claim 51, wherein the activator emits radiation to drive dissociation, wherein the emitted radiation is between about 180 to 640 nm.

55. The system of claim 51, wherein the activator provides a dissociation voltage is between about 1 to 20 volts.

56. The system of claim 51, wherein the dissociation voltage is between about 5 to 20 volts.

57. The system of claim 51, wherein the dissociation voltage is between about 5 to 12 volts.
